(19) United States
(12) Patent Application Publication
(10) Pub. No.: US 2021/0052772 A1
(43) Pub. Date: Feb. 25, 2021

(54) IMPLANTABLE DEVICE COMPRISING A TEXTILE COMPONENT COMPRISING MULTIFILAMENT YARNS AND/OR SPUN YARNS MADE OF FIBERS COMPLETELY OR PARTIALLY COATED IN CYCLODEXTRIN POLYMER

(71) Applicant: COUSIN BIOTECH, WERVICQ-SUD (FR)

(72) Inventors: Guillaume VERMET, LYON (FR); Bernard MARTEL, NIEPPE (FR); Nicolas BLANCHEMAIN, MERVILLE (FR); Stéphanie DEGOUTIN, LANDAS (FR)

(21) Appl. No.: 17/050,063

(22) PCT Filed: Apr. 5, 2019
(86) PCT No.: PCT/FR2019/050803

§ 371 Date: Oct. 23, 2020

(30) Foreign Application Priority Data
Apr. 27, 2018 (FR) 18 53737

(51) Int. Cl. A61L 27/34; A61L 27/16; C08L 5/16; A61L 27/54; D02G 3/44; D02G 3/04; D01F 8/14; D01F 8/16

(52) U.S. Cl. CPC A61L 27/34; A61L 27/16; C08L 5/16; A61L 27/54; D02G 3/448; A61L 2300/402; D01F 8/14; D01F 8/16; A61L 2420/02; A61L 2420/06; A61L 2300/406; D02G 3/045

(57) ABSTRACT

The present invention relates to an implantable device comprising a textile component at least partially coated with a host polymer coating, said host polymer coating comprises a polymer of cyclodextrin(s) and/or derivatives of cyclodextrin(s) and/or inclusion complex(es) of cyclodextrin and/or inclusion complex(es) of cyclodextrin derivatives. Advantageously, said textile component comprises multifilament yarns and/or spun yarns made of fibers, at least a part of the filaments and/or fibers each having a diameter less than or equal to 25 micrometers.

### BACKGROUND OF THE INVENTION

The present invention relates to the technical field of implantable devices comprising a textile component comprising multifilament yarns and/or spun yarns made of fibers at least partially coated with a cyclodextrin polymer.

Implantable devices comprising a textile component can be used in numerous applications, for example in the spine, for the treatment of abdominal hernia, as a ligament replacement, for the treatment of pelvic organ prolapse (for example in the treatment of urinary incontinence), for the treatment of male erectile dysfunction, for the treatment of obesity; and in a wide variety of forms: flat or three-dimensional components, for example in the form of a dome, a wedge, a braid, or strips.

Following the placement of an implantable device, complications, such as adhesions, pain, and post-operative infections, may occur and delay healing and the patient's return to normal activity, particularly in the treatment of abdominal hernias.

Furthermore, post-operative pain in the first few days following a surgical procedure has recently been linked to the development of chronic pain several months after surgery, resulting in morbidity that may require additional treatment.

Such consequences can be prevented by prophylactic treatment consisting of local injection of morphine derivatives or continuous instillation of local anesthetics after implantation.

However, a number of protocols for post-operative analgesia by direct infiltration into the surgical site or continuous instillation of local anesthetics have been proposed without reaching a satisfactory compromise between the method of pain treatment and effective pain relief. Indeed, the expected and long-lasting analgesic effect cannot be obtained due to the rapid elimination of the medicinal product from the operated site by the circulating flow of body fluids.

Another approach consists in modifying implantable devices so as to give them properties of delivery of active agent(s), such as analgesics, in situ and in a relatively prolonged manner depending on the delivery device selected.

These in situ delivery devices include cyclodextrin polymers applied to a textile to form a host coating that will be functionalized by the addition of one or more guest active agent(s). For example, WO 2006/051227 A1 describes the manufacture of a biomaterial which can be made of textile and comprising a cyclodextrin polymer. The complexed active agent is chosen according to its ability to be complexed with cyclodextrin cage molecules and according to the desired therapeutic activity. It can thus be an antibiotic in order to avoid any bacterial contamination of the implanted prosthesis.

The publication entitled “Visceral mesh modified with cyclodextrin for the local sustained delivery of ropivacaine”, International Journal Of Pharmaceutics 476 (2014) 149-159 (authors: G. Vermet, S. Degoutin, F. Chai, M. Maton, M. Bria, C. Danel, H. F. Hildebrand, N. Blanchemain, B. Martel) also describes an in situ delivery device applied to a polyester textile plate comprising a ropivacaine complexing cyclodextrin polymer to reduce post-operative pain.

Generally, textiles are used as implantable materials because their flexibility allows them to adapt to complex shapes, particularly the anatomical shape of organs.

Depending on the desired properties, the textiles can be rigid or semi-rigid or flexible. These implantable textiles can be rolled up on themselves when they are flexible and in the form of flat panels, can follow complex anatomical paths when they are in the form of braids, or can perform specific functions such as filling according to their 3D configuration.

Implantable textile components comprising monofilament yarns are preferred because they are considered less conducive to the development of bacterial infection compared with implantable textile components comprising multifilament yarns (see J. Conze, R. Rosch, U. Klinge, C. Weiss, M. Anurov, S. Titkowa, A. Oettinger, V. Schumpelick, Polypropylene in the intra-abdominal position: influence of pore size and surface area, Hernia 8 (2004) 365-372). However, it is difficult to attach a polymer coating to these textile components consisting of monofilament yarns.

Advantageously, textile components comprising multifilament yarns have a higher specific surface area and porosity than those of textile components comprising monofilament yarns as well as more interfilament spaces confined to the core of the yarn which can be filled with a polymer coating.

In addition, the deposition of a polymer coating on a textile, depending on its nature, can modify the flexibility of the textile to a point where it can no longer properly provide its primary functions. This is the case for example with abdominal wall repair prostheses when the prosthesis can no longer be rolled up on itself to be inserted in a trocar (in particular having a diameter of the order of 10 mm or is too thick in the rolled up state).

This parameter may be prohibitive for the use of a functionalized implantable textile component since, for the host polymer coating to be effective, it must be deposited in sufficient amount on the textile component in order to absorb an amount of active agent(s) that corresponds to a delivered therapeutic dose which is effective but non-toxic. This absorbed dose of active agent must therefore be within the therapeutic window, namely between a minimum value (efficacy threshold) and a maximum value (toxicity threshold).

The present invention thus has as its object an implantable device comprising a textile component at least partially coated with a cyclodextrin polymer retaining its intrinsic mechanical properties, in particular its flexibility.

The present invention also has as its object an implantable device whose duration and intensity of the effects associated with one or more complexed active agent(s) (for example analgesic and/or antibacterial) are improved.

### OBJECT AND SUMMARY OF THE INVENTION

The present invention has as its object, according to a first aspect, an implantable device, overcoming the above-mentioned problems, comprising a textile component comprising multifilament yarns and/or spun yarns made of fibers at least partially coated with a host polymer coating, said host polymer coating comprises a polymer of cyclodextrin(s) and/or derivatives of cyclodextrin(s) and/or inclusion complex(es) of cyclodextrin and/or inclusion complex(es) of cyclodextrin derivatives. Advantageously, at least a part of the filaments and/or fibers each have a diameter less than or equal to 25 micrometers (μm).

Despite the biases of persons skilled in the art, the inventors found that the combination of a host polymer coating with a textile component having filaments and/or fibers of a certain fineness makes it possible to obtain a flexible implant that can be bent without the coating becoming detached from the fiber or filament when the implant is handled. This combination also makes it possible to improve the grafting rate of the host polymer and thus to further improve the desired therapeutic effect due to the improved loading of the active agent(s).

Advantageously, textile components comprising multifilament yarns, and/or spun yarns made of fibers, in particular multifilament yarns, have a higher specific surface area and porosity than textile components comprising monofilament yarns, as well as more interfilament spaces confined to the core of the yarn that can be filled with a polymer coating.

In the present text, a textile component is understood to be any component obtained by the use of at least one yarn, in particular said yarn(s) may be a multifilament yarn and/or a spun yarn made of fibers.

The textile component according to the invention comprises multifilament yarns but may also comprise spun yarns made of fibers, although this is not preferred.

The density of the polyethylene terephthalate is considered to be preferably of the order of 1.38 g/cm³ and that of the polypropylene is considered to be preferably of the order of 0.9 g/cm³.

The diameter of a filament or fiber is preferably calculated by considering that the filament or fiber is a perfect cylinder from the following formula:

diameter of the filament or fiber [μm]=20*Root²(Titer [dtex]) filament or fiber/(density of the material constituting the fiber or filament [g/cm³]×1/3.14)).

The textile component according to the invention may be a knitted element, in particular a weft knit or warp knit, a woven element, a braided element, a twisted or cabled element, or resulting from their different combinations.

A multifilament yarn consists of a set of several filaments. The multifilament yarn is mechanically handled on a textile manufacturing machine, such as a knitting, weaving or braiding machine.

The fibers according to the invention can be in the form of a spun yarn made of fibers.

Preferably, the textile component is in a textile material comprising opposite first and second faces, again preferably the host polymer coating covers all or part of the first face and/or the second face.

In an embodiment, a part or all of the filaments of the multifilament yarns and/or the fibers of the spun yarns made of fibers have a diameter of less than or equal to 20 μm, preferably less than or equal to 18 μm, more preferably less than or equal to 17 μm, in particular greater than or equal to 5 μm.

In an embodiment, the mass of the host polymer coating (mh), in particular its surface density (g/m²), relative to the total mass (g) (mt), in particular the total surface density (g/m²), of the textile component comprising the host polymer coating (mh/mt *100), is greater than 0% and less than or equal to 60%, preferably less than or equal to 50%, in particular less than or equal to 40%, in particular greater than or equal to 5%, more particularly greater than or equal to 10%.

In an embodiment, the grafting rate of host polymer coating, in particular of cyclodextrin polymer defined according to the invention, is greater than or equal to 10%, preferably greater than or equal to 15%, more preferably greater than or equal to 20%, in particular greater than or equal to 25%, more particularly greater than or equal to 30%.

The grafting rate of host polymer coating can be less than or equal to 60%.

The grafting rate is calculated as the mass gain (expressed as a percentage) of the textile component in host polymer coating. This grafting rate is equal to: ((mass textile component including host polymer coating)−(mass virgin textile component i.e. without host polymer coating))/(mass virgin textile component i.e. without host polymer coating)*100.

In the context of the present invention, the mass of the virgin textile component or textile component coated with the host polymer coating is preferably measured after drying at 104° C. for one hour (and optionally after the neutralization step and/or the washing step defined below) and placing it in a desiccator at room temperature (of the order of 20° C.-25° C.) for at least 30 minutes.

In an embodiment, at least a part, in particular all, of the spun yarns made of fibers and/or multifilament yarns each have a titer of less than or equal to 500 dtex, preferably less than or equal to 400 dtex, more preferably less than or equal to 300 dtex, more preferentially less than or equal to 200 dtex, in particular less than or equal to 100 dtex.

In an embodiment, at least a part of the filaments and/or fibers of the yarns, in particular the filaments of the multifilament yarns, each have a titer less than or equal to 10 dtex (and implicitly greater than 0 dtex), preferably less than or equal to 5 dtex, more preferably less than or equal to 4 dtex, in particular less than or equal to 3 dtex.

In an embodiment, the textile component (excluding the host polymer coating) has an surface density greater than or equal to 10 g/m² and less than or equal to 500 g/m², preferably less than or equal to 300 g/m², more preferably less than or equal to 120 g/m², preferentially less than or equal to 80 g/m², more preferentially less than or equal to 60 g/m², in particular less than or equal to 40 g/m², for example of the order of 38 g/m².

In an embodiment, the textile component is flat or three-dimensional, preferably the textile component has a substantially plate-like shape, possibly smooth or in relief.

The cyclodextrin polymer present on the textile component is formed by a crosslinking reaction between cyclodextrin or a cyclodextrin derivative and at least one crosslinking agent defined below. Cyclodextrin and/or a cyclodextrin derivative and the at least one crosslinking agent are mixed in an aqueous solution and then the textile component is impregnated with said aqueous solution, for example by padding. This impregnation operation, in particular by padding, allows the cyclodextrin and/or cyclodextrin derivative and the at least one reactive agent to penetrate by capillary action into the interstices formed between the filaments of the yarns and/or between the fibers. The cyclodextrin and/or cyclodextrin derivative polymer thus fills the interfilament and/or interfiber space and the outer surface of the yarn and/or fibers.

Advantageously, the yarns and/or fibers according to the invention have a high specific surface area and rate of absorption of the aqueous solution during impregnation. The grafting rate of cyclodextrin polymer and/or cyclodextrin derivative is thus improved. Surprisingly, the textile component retains its intrinsic flexibility.

In the present text, “cyclodextrin” means any native cyclodextrin, in particular alpha-cyclodextrin or beta-cyclodextrin or gamma-cyclodextrin.

In the present text, “cyclodextrin derivative” means any native cyclodextrin, in particular alpha-cyclodextrin or beta-cyclodextrin or gamma-cyclodextrin, of which one or more hydroxyl group(s), is/are substituted, in particular aminated, in particular by an amine function (—NH₂), esterified, alkylated, hydroxyalkylated, carboxyalkylated, in particular carboxymethylated, sulfoalkylated, in particular sulfobutylated.

Said at least one hydroxyl group of the cyclodextrin may be substituted by a linear and/or branched, unsaturated or unsaturated, substituted or unsubstituted alkyl chain having from 1 to 10 carbon atoms, preferably from 1 to 6 carbon atoms, more preferably from 1 to 4 carbon atoms, in particular a methyl group, an ethyl group, an n-propyl group, an isopropyl group, an n-butyl group, an iso-butyl group, a sec-butyl group, a tert-butyl group, each of said groups being optionally, and independently of one another, substituted by one or more hydroxyl groups (—OH); and/or substituted by one or more carboxylic acid groups (—COOH); and/or substituted by one or more sulfonic acid groups and/or one or more salts of said sulfonic and carboxylic acid functions. Said at least one hydroxyl group of the cyclodextrin may thus be substituted by a hydroxypropyl group or by a carboxymethyl group.

In the present text, “inclusion complex of cyclodextrin” or “inclusion complex of cyclodextrin derivative” means any cyclodextrin or cyclodextrin derivative within the meaning of the present invention complexing a functional agent as defined in the present text.

The skilled person selects one or more cyclodextrin(s) and/or one or more cyclodextrin derivative(s) and/or one or more cyclodextrin (derivative) inclusion complex(es) capable of reacting with the crosslinking agent. In particular, if one of more cyclodextrin derivative(s) is/are used, it/they must have sufficient hydroxyl groups (—OH) to be capable of carrying out a polycondensation reaction with the corresponding (poly)carboxylic acids and/or their corresponding anhydrides.

EP 1 165 621 B1 describes the synthesis of cyclodextrin polymers obtained by polycondensation reactions between (poly)carboxylic acids and native cyclodextrins (alpha, beta, gamma) or derived cyclodextrins (methyl, hydroxypropyl cyclodextrins) suitable for the implementation of the invention.

They are characterized by a high cyclodextrin content, comprised between 40% and 65% by mass and by its richness in carboxylic acid functions, of the order of 3-5 mmol per gram of cyclodextrin polymer.

For the purposes of the present invention, “carboxylic acid” means an acid containing a —COOH function.

For the purposes of the present invention, “polycarboxylic acid” means an acid comprising at least two —COOH functions, and “its corresponding anhydride” means an anhydride formed from the condensation of two carboxylic acid functions, in particular comprising at least one —CO—O—CO- function.

In a variant, the ratio of the mass of the host coating to the total mass of the textile component comprising the host coating is greater than or equal to 10%, preferably greater than or equal to 12%, and more preferably greater than or equal to 15%.

In a sub-variant, this ratio is greater than or equal to 20%, in particular greater than or equal to 25%.

In a variant, the textile component (excluding the host polymer coating) has a surface density less than or equal to 120 g/m², preferably less than or equal to 80 g/m², more preferably less than or equal to 70 g/m², more preferably less than or equal to 50 g/m².

In a variant, the textile component comprises, preferably in a proportion by mass of at least 80% in relation to its total mass, more preferably in a proportion by mass of at least 95% in relation to its total mass, multifilament yarns and/or spun yarns made of fibers with a titer of each filament of less than or equal to 10 dtex, preferably less than or equal to 7 dtex, more preferably less than or equal to 4 dtex.

In a variant, the textile component is a knitted component, especially with warp stitches.

Preferably, the textile component is a locknit material.

In a variant, the device according to the invention is chosen from the list consisting of a prosthesis for the treatment of a hernia in the abdominal region, in particular an inguinal, femoral, umbilical or epigastric hernia; a ligament; a prosthesis for the treatment of a prolapse of at least one pelvic organ; a prosthesis for the treatment of male erectile dysfunction; a prosthesis for the treatment of the spine, in particular an intervertebral prosthesis; preferably a prosthesis for the treatment of a hernia in the abdominal region.

In a variant, the multifilament yarns and/or spun yarns made of fibers are made of at least one or more rebsorbable, partially rebsorbable or non-resorbable polymer(s), in particular in one or more polymer(s) chosen from List I consisting of: polyolefins, and in particular polypropylene and polyethylene; polymers based on polyethylene oxide (PEO), block copolymers based on polypropylene oxide and ethylene oxide, in particular of the diblock or triblock type, such as PPO-PEO (i.e. poly(propylene oxide-ethylene oxide), PPO-PEO-PPO (i.e. poly(propylene oxide-ethylene oxide-propylene oxide) or PEO-PPO-PEO (i.e. poly(ethylene oxide-propylene oxide-ethylene oxide)), polyesters (in particular non-rebsorbable polyesters), and in particular polyethylene terephthalate; polyamides, and in particular polyamide 6-6; more preferably it is polypropylene or polyethylene terephthalate; and/or in List II consisting of: (co)polymers of lactic acid, derived from the polymerization of at least L-lactide and/or at least D-lactide and/or at least meso-lactide; polymers derived from the polymerization of at least glycolide, for example polyglycolic acid (PGA); copolymers derived from the polymerization of at least lactide and at least glycolide, for example PLGA (polylactide-co-glycolide); block (co)polymers based on polylactide and polyethylene glycol, for example of the PLA-PEG-PLA type; or a mixture thereof.

The lactic acid (co)polymers defined above are commonly referred to generically as polylactic acid (PLA) polymers.

In a variant, the host polymer coating comprises at least one guest functional agent selected from the list comprising: antibiotics, for example ciprofloxacin; anti-inflammatory agents; anticoagulants; anti-thrombogenic agents; anti-mitotic agents; anti-proliferation agents; anti-adhesion agents; anti-migration agents; cell adhesion promoters; growth factors; antiparasitic molecules; hormones; antifungals; antimicrobial molecules; antiseptics; analgesic agents such as ropivacaine, bupivacaine, lidocaine, levobupivacaine, preferably ropivacaine.

In a variant, the textile component comprises openings, in particular comprises openings with at least one dimension greater than or equal to 1 mm.

These openings are preferably through-going, i.e. they pass through the thickness of the textile material forming the textile component and open on its first and second faces.

These openings are preferably delimited between the yarns, in particular in a repeated manner according to the mesh pattern or the weave for example.

Advantageously, the textile component allows the development of fibrosis and its integration in living tissues by means of these openings.

In a variant, the textile component is chosen from the list consisting of: knitted or weft knitwear; fabrics, braids, twisted elements, or a combination thereof.

The present invention has as its object, according to a second aspect, a process for manufacturing an implantable device, preferably according to any one of the embodiment variants with reference to a first aspect of the invention, comprising the following steps:
(i) providing a textile component comprising multifilament yarns and/or spun yarns made of fibers, at least a portion of the filaments and/or fibers each having a diameter less than or equal to 25 micrometers (μm);
(ii) applying an aqueous solution comprising:
   at least one cyclodextrin and/or at least one cyclodextrin derivative and/or at least one inclusion complex of cyclodextrin and/or at least one inclusion complex of cyclodextrin derivative;
   at least one (poly)carboxylic acid and/or its (poly)carboxylic acid anhydride,
   and optionally at least one catalyst;

on at least a part of the multifilament yarns and/or spun yarn made of fibers;
(iii) applying a heat treatment to the textile component obtained after step (ii) in order to form a host coating comprising a polymer of cyclodextrin(s) and/or derivatives of cyclodextrin(s) and/or inclusion complex(es) of cyclodextrin and/or inclusion complex(es) derivatives of cyclodextrins;
obtaining the implantable device.

Preferably, the aqueous solution is applied by padding.

In a variant, the (poly)carboxylic acid or its corresponding acid anhydride is selected from the list comprising: saturated or unsaturated or aromatic, linear or branched or cyclic (poly)carboxylic acids and hydroxypoly(carboxylic) acids, preferably from the list comprising: citric acid, polyacrylic acid, poly(methacrylic acid), 1,2,3,4-butanetetracarboxylic acid (BTCA), maleic acid, citraconic acid, itaconic acid, 1,2,3-propanetricaboxylic acid, trans-aconitic acid, all-cis-1,2,3,4-cyclopentanetetracarboxylic acid, mellitic acid, pyromellitic acid, ethylenediamine tetraacetic acid (EDTA), oxydisuccinic acid, thiodisuccinic acid or from the acid anhydrides of the aforesaid (poly)carboxylic acids, such as pyromellitic dianhydride, and mixtures thereof, preferably citric acid and BTCA.

In an embodiment, the heat treatment in step (iii) comprises the application of a temperature greater than or equal to 80° C., preferably a temperature greater than or equal to 90° C., more preferably greater than or equal to 100° C., in particular greater than or equal to 130° C., for at least one minute, more preferably for at least 5 minutes.

In an embodiment, the heat treatment in step (iii) comprises a first step (iii-a) during which heating is carried out at a given temperature so as to evaporate the water, in particular without inducing polymerization, and a second step (iii-b) during which heating is carried out at a given temperature so as to cause the host polymer coating to polymerize.

Preferably, the temperature during the first step (iii-a) is greater than or equal to 90° C., preferably for at least ten minutes.

Preferably, the temperature during the second step (iii-b) is greater than or equal to 120° C., more preferably greater than or equal to 135° C., in particular for at least thirty minutes, in particular for at least two hours.

The temperature applied during steps (iii-a) and (iii-b) is lower than the degradation temperature of the textile component, preferably less than or equal to 250° C., and preferably less than or equal to 180° C.

The application time during steps (iii-a) and (iii-b) is preferably less than or equal to 360 minutes.

Preferably, the manufacturing process comprises a step of washing (iv) of the textile component after step (iii) (in particular after step (iii-b) and/or the neutralization step below) during which the textile component is washed with water, in particular distilled water or pure water, or an aqueous solution, having a temperature greater than or equal to 45° C., in particular greater than or equal to 70° C.

This operation removes the reagents present in the aqueous solution which have not acted.

In a variant, the process comprises a step of neutralizing the residual acidity of the host coating, preferably a basic solution, in particular a solution of sodium carbonate or sodium hydrogen carbonate (bicarbonate), is applied to the textile component obtained after step (iii) (in particular step (iii-b)).

Preferably, an intermediate wash (similar to the wash in step (iv)) is performed after step (iii) (in particular (iii-b) and before the neutralization step, again preferably before step (iv).

This treatment with a weak base causes the conversion of the residual carboxylic acid functions of the cyclodextrin polymer coating to carboxylate functions. Advantageously, the inventors have surprisingly found that this step can make the textile component coated with the host polymer more flexible.

This step also improves the cytocompatibility of the implantable device.

Before implantation, the implantable device is sterilized, for example in its packaging bag by irradiation or with ethylene oxide.

### DETAILED DESCRIPTION OF THE INVENTION

The present invention will be better understood by reading the examples of embodiments described below and cited on a non-limiting basis.

### Materials Used:

Textile component A: 38 g/m² flat warp-knitted panel consisting of multifilament yarns of polyethylene terephthalate, said yarns each having a titer of 61 dtex and each comprising 24 filaments (or about 2.54 dtex/filament, or about a diameter of 15.3 μm/filament).

Textile component B: 100 g/m² flat warp-knitted panel consisting of multifilament yarns of polyethylene terephthalate, the warp yarns (70% in number), in particular forming the warp, each having a titer of 138 dtex and each comprising 32 filaments (or about 4.31 dtex, or about a diameter of 20 μm/filament); and the weft yarns (30% in number) each having a titer of 226 dtex, and each comprising 64 filaments (or about 3.53 dtex, or about a diameter of 18 μm/filament). The average diameter of the filaments is thus about 19.5 μm.

Hydroxypropyl-β-cyclodextrin (HPβD), of the brand Kleptose® HPB, MS=0.62; M=1387 g/mol from Roquette Freres (Lestrem, France).

Citric acid (CTR), sodium dihydrogen hypophosphite (NaH₂PO₄.xH₂O) and ropivacaine (M=274 g/mol=from Aldrich Chemicals (Saint Quentin Fallavier, France).

Ropivacaine hydrochloride solution: Naropin® 2 mg/ml and Ropivacaine KABI 10 mg/ml, respectively from AstraZeneca (London, England) and Kabi (Bad Homburg, Germany).

Textile components A and B are first washed by Soxhlet extraction with diethyl ether (24 cycles, one hour per cycle) with the goal of removing lubricants and possible residual compounds, for example sizing agents.

### Adsorption/Desorption of Ropivacaine

After functionalization of the textile components by the cyclodextrin polymer, the textile components are impregnated in a ropivacaine hydrochloride solution at room temperature (of the order of 20-25° C.). In order to evaluate the amount of ropivacaine hydrochloride adsorbed by the textile components, the adsorbed ropivacaine is desorbed in 10 ml of water and then in 5 ml of 0.1M soda NaOH. The desorption solutions obtained are analyzed using a high-performance liquid chromatography method (LC 2010A-HT, Shimadzu, Noisiel, France, equipped with a visible-ultraviolet detector and a Dell integrator/recorder computer with LabSolutions software). The chromatograph was adapted with a Gemini-NX C18 column (5 μm, 250*4.6 mm, Phenomenex, Le Pecq, France). The mobile phase consists of a mixture of a 72% phosphate buffer solution (pH 2.5) and 28% acetonitrile. Chromatographic separation was performed at 25° C. with a flow rate of 1 mL/min and controlled at 201 nm. The approximate retention time for ropivacaine at 20 nm is 5.5 minutes under these conditions.

The amount of ropivacaine desorbed in ultrapure water corresponds to the ropivacaine adsorbed by capillary action on the textile component itself, “independently” of the host polymer coating (non-specific interactions). The amount of ropivacaine desorbed in 0.1M soda NaOH corresponding to the ropivacaine loaded by the host polymer coating (specific interactions with the cyclodextrin polymer: inclusion complexes, ionic bonds hydrogen bonds).

### EXAMPLE 1

Textile component A is impregnated and padded (2 m/min, 2 bar, Laboratory Padder LDP, LAB-PRO, Wikon, Switzerland) in an aqueous solution comprising HPβCD, a catalyst (NaH₂PO₂) and CTR, in the following proportions 8/1/10 where 8, 1 and 10 are the masses (grams) of HPβCD, catalyst (NaH₂PO₂) and CTR, respectively, dissolved in 100 mL ultrapure water. The impregnated textile component A is placed in a heat-setting chamber (Model FD, BINDER, Tuttlingen, Germany) for 5 minutes at 90° C. and then for 120 minutes at 140° C. The coated textile component A is then washed twice rapidly for 3 minutes in ultrapure water and then neutralized by passage in an aqueous solution containing sodium carbonate at 4 g/L in order to neutralize the residual acidity. The textile component is then rinsed twice quickly in ultrapure water for 3 minutes. The textile component is then washed in ultrapure water (washing step) at 80° C. (FISONS Cl, Thermo Scientific Haake, Karlsruhe, Germany) for 20 minutes and dried at 104° C. for 1 hour, and finally cooled to room temperature in a desiccator for 30 minutes. The host coating grafting rate is of the order of 13.9%. The final textile component A is flexible and wraps around itself without altering its shape or detaching from the CD polymer coating.

### EXAMPLE 2

Textile component A is impregnated and padded (2 m/min, 2 bar, Laboratory Padder LDP, LAB-PRO, Wikon, Switzerland) in an aqueous solution comprising HPβCD, catalyst (NaH₂PO₂) and CTR, in the following proportions 12/1.5/15 where 12; 1.5 and 15 are the masses (grams) of HPβCD, catalyst (NaH₂PO₂) and CTR, respectively, dissolved in 100 mL ultrapure water. The impregnated textile component A is placed in a heat-setting chamber (Model FD, BINDER, Tuttlingen, Germany) for 5 minutes at 90° C. and then for 120 minutes at 140° C. The coated textile component A is then washed twice rapidly for 3 minutes in ultrapure water and then neutralized by passage in an aqueous solution containing sodium carbonate at 4 g/L in order to neutralize the residual acidity. The textile component is then rinsed twice quickly in ultrapure water for 3 minutes. The textile component is then washed in ultrapure water at 80° C. (FISONS Cl, Thermo Scientific Haake, Karlsruhe, Germany) for 20 minutes and dried at 104° C. for 1 hour, and finally cooled to room temperature in a desiccator for 30 minutes. The host coating grafting rate is of the order of 21%. The final textile component A is flexible and wraps around itself without altering its shape or detaching from the host polymer CD coating.

### EXAMPLE 3

Textile component A is impregnated and padded (2 m/min, 2 bar, Laboratory Padder LDP, LAB-PRO, Wikon, Switzerland) in an aqueous solution comprising HPβCD, a catalyst (NaH₂PO₂) and CTR, in the following proportions 16/2/20 where 16, 2 and 20 are the masses (grams) of HPβCD, catalyst (NaH₂PO₂) and CTR, respectively, dissolved in 100 mL of pure water. The impregnated textile component A is placed in a heat-setting chamber (Model FD, BINDER, Tuttlingen, Germany) for 5 minutes at 90° C. and then for 120 minutes at 140° C. The coated textile component A is then washed twice rapidly for 3 minutes in ultrapure water and then neutralized by passage through an aqueous solution containing concentrated sodium carbonate at 4 g/L in order to neutralize the residual acidity. The textile component is then rinsed twice quickly in ultrapure water for 3 minutes. The textile component is then washed in ultrapure water at 80° C. (FISONS Cl, Thermo Scientific Haake, Karlsruhe, Germany) for 20 minutes and dried at 104° C. for 1 hour, and finally cooled to room temperature in a desiccator for 30 minutes. The host coating grafting rate is of the order of 29%. The final textile component A is flexible and wraps around itself without altering its shape or detaching from the host polymer CD coating.

### EXAMPLE 4A

An 11 mm diameter disc cut from textile component A obtained at the end of Example 1 is immersed in a ropivacaine hydrochloride solution at 2 mg/mL for 5 minutes, and then immersed for 60 minutes in 10 ml of ultrapure water to remove unadsorbed ropivacaine. The amount (g) of desorbed ropivacaine relative to the total mass of the textile component A coated with the CD polymer is 9.0±2.5 mg/g.

### EXAMPLE 4B

An 11 mm diameter disc cut from textile component A obtained at the end of Example 1 is immersed in a 10 mg/mL ropivacaine hydrochloride solution for 5 minutes, and then immersed for 60 minutes in 10 ml ultrapure water to remove unadsorbed ropivacaine. The amount (g) of desorbed ropivacaine relative to the total mass of the textile component A coated with the CD polymer is 60 mg/g.

### EXAMPLE 5A

same protocol as for Example 4A but applied to Example 2.

### EXAMPLE 5B

same protocol as for Example 4B but applied to Example 2.

### EXAMPLE 6A

same protocol as for Example 4A but applied to Example 3.

### EXAMPLE 6B

same protocol as for Example 4B but applied to Example 3.

For Examples 5A and 6A, desorption rates in ultrapure water are similar to the ropivacaine desorption rate in ultrapure water obtained for Example 4A. The same applies to the desorption rates in ultrapure water in Examples 5B and 6B, which are similar to the ropivacaine desorption rate in ultrapure water in Example 4B.

### EXAMPLE 7A

The disc obtained at the conclusion of Example 4A is immersed in 5 ml of sodium hydroxide solution at 0.1M for 5 minutes at 37° C. to completely hydrolyze the host polymer coating formed from HPβCD, thus completely releasing the adsorbed ropivacaine. The amount (g) of desorbed ropivacaine relative to the total mass of the textile component A coated with the CD polymer is 5.1±0.1 mg/g.

### EXAMPLE 7B

The disc obtained at the conclusion of Example 4B is immersed in 5 ml of sodium hydroxide solution at 0.1M for 5 minutes at 37° C. to completely hydrolyze the host polymer coating formed from HPβCD, thus completely releasing the adsorbed ropivacaine. The amount (g) of desorbed ropivacaine relative to the total mass of the textile component A coated with the CD polymer is 18.8±0.1 mg/g. The total amount of ropivacaine desorbed in water and soda is therefore of the order of 78.8 mg/g of support, i.e. of textile component A.

### EXAMPLE 8A

same protocol as for Example 7A but applied to Example 5A. The amount (g) of desorbed ropivacaine relative to the total mass of the textile component A coated with the CD polymer is 7.2±0.5 mg/g.

### EXAMPLE 8B

same protocol as for Example 7B but applied to Example 5B. The amount (g) of desorbed ropivacaine relative to the total mass of textile component A coated with CD polymer is 23.1±1.1 mg/g.

### EXAMPLE 9A

same protocol as for Example 7A but applied to Example 6A. The amount (g) of desorbed ropivacaine relative to the total mass of textile component A coated with CD polymer is 9.4±0.8 mg/g.

### EXAMPLE 9B

same protocol as for Example 7B but applied to Example 6B. The amount (g) of desorbed ropivacaine relative to the total mass of the textile component A coated with CD polymer is 33.9±1.1 mg/g.

### EXAMPLE 10

Textile component B is impregnated and padded in an aqueous solution comprising HPβCD, a catalyst (NaH₂PO₂) and CTR, in the following proportions 8/1/10 where 8, 1 and 10 are the masses (in grams) of HPβCD, catalyst (NaH₂PO₂) and CTR, respectively, dissolved in 100 mL of ultrapure water. The impregnated textile component B is placed in a heat-setting chamber (Minithermo®, Roaches, UK) for 60 minutes at 160° C. The coated textile component B is washed with distilled water and then treated in an aqueous solution containing sodium carbonate, then washed with distilled water at a temperature of the order of 80° C. in a Soxhlet extractor. The host coating grafting rate is of the order of 13.6%.

### EXAMPLE 11

Textile component B is impregnated and padded in an aqueous solution comprising HPβCD, a catalyst (NaH₂PO₂) and CTR, in the following proportions 12/1.5/15 where 12; 1.5 and 15 are the masses (in grams) of HPβCD, catalyst (NaH₂PO₂) and CTR, respectively, dissolved in 100 mL of ultrapure water. The impregnated textile component B is placed in a heat-setting chamber (Minithermo®, Roaches, UK) for 60 minutes at 160° C. The coated textile component B is washed with distilled water and then treated in an aqueous solution containing sodium carbonate, then washed with distilled water at a temperature of the order of 80° C. in a Soxhlet extractor. The host coating grafting rate is of the order of 17.4%.

### EXAMPLE 12

Textile component B is impregnated and padded in an aqueous solution comprising HPβCD, a catalyst (NaH₂PO₂) and CTR, in the following proportions 16/2/20 where 16, 2 and 20 are the masses (in grams) of HPβCD, catalyst (NaH₂PO₂) and CTR, respectively, dissolved in 100 mL of ultrapure water. The impregnated textile component B is placed in a heat-setting chamber (Minithermo®, Roaches, UK) for 60 minutes at 160° C. The coated textile component B is washed with distilled water and then treated in an aqueous solution containing sodium carbonate, then washed with distilled water at a temperature of the order of 80° C. in a Soxhlet extractor. The host coating grafting rate is of the order of 27.7%.

### EXAMPLE 13

An 11 mm disc of textile component B of Example 11 undergoes the protocols described in Examples 4A and 7A. The rate of desorbed ropivacaine in a 0.1M soda NaOH solution is of the order of 4 mg/g.

### COMPARATIVE EXAMPLE 14

An 11 mm disc of a virgin textile component B undergoes the protocols described in Examples 4A and 7A. The rate of desorbed ropivacaine in a 0.1M soda NaOH solution is of the order of 0.5 mg/g (from textile component B). It should be noted that the same behavior, i.e. very low ropivacaine absorption, is observed for the virgin textile component A.

### EXAMPLE 15

An 11 mm disc of textile component B coated with CD in Example 11 undergoes the protocols described in Examples 4B and 7B (The amount of ropivacaine desorbed in a 0.1M soda NaOH solution is of the order of 9 mg/g). The total amount of ropivacaine desorbed (in water and soda) is of the order of 49.4 mg/g of textile component B.

### COMPARATIVE EXAMPLE 16

An 11 mm disc of a virgin textile component B undergoes the protocols described in Examples 4B and 7B. The rate of desorbed ropivacaine in a 0.1M soda NaOH solution is of the order of 2 mg/g. It should be noted that the same behavior, i.e. very low ropivacaine absorption, is observed for the virgin textile component A.

The total amount of ropivacaine adsorbed (mg/g of support) by the functionalized textile component A (Example 7B) is increased by 60% compared with the functionalized textile component B (Example 15).

If the total amount of ropivacaine per m² of textile component is considered, the amount of ropivacaine adsorbed (mg/m² of textile support) for textile component B in Example 15 is of the order of 5611 mg/m² versus 3410 mg ropivacaine per m² of textile support A in Example 7B. Thus, although textile component A is 2.6 times lighter than textile component B, and the amount of ropivacaine adsorbed per m² is 0.39 times less (5.28 g of grafted CD polymer versus 13.6 g of grafted CD polymer), the amount of adsorbed ropivacaine is only 0.60 times less.

This advantage results in a flexible prosthesis with improved active agent absorption capabilities. Thus, the use of a lighter textile support offers an excellent compromise between i) the amount of cyclodextrin polymer immobilized per m² of textile component, ii) the amount of ropivacaine loaded on the textile component, and iii) the flexibility of the textile component.

The amount of analgesic loaded by the textile component A or B functionalized by the CD polymer is higher than that loaded by a virgin textile component A or B, in particular when the ropivacaine solution is concentrated at 10 mg/mL.

The presence of the CD polymer would thus improve the amount of ropivacaine physically adsorbed by the textile component.

The textile components according to the invention were tested to determine their effectiveness in the treatment of pain based on the colorectal distension model developed by Rousseaux et al. (“Lactobacillus acidophilus modulates intestinal pain and induces opioid and cannabinoid receptors. Nat. Med. 13 (2007) 35-37) and Yang et al. (”Establishment of model of visceral pain due to colorectal distension and its behavioral assessment in rats. World J. Gastroenterol. 12 (2006) 2781-2784). Textile components A, virgin and functionalized with a host polymer coating according to Example 3, not loaded with ropivacaine, were implanted in two groups of ten rats each. Pain was evaluated one day after implantation up to 8 days. No post-operative deaths were observed.

A low pain threshold of 38 mmHg (versus 46 mmHg considered as the baseline level) is observed for the virgin textile component A and the functionalized textile component A according to Example 3 not loaded with ropivacaine, one day after implantation. This low pain tolerance reveals the surgically induced visceral pain and the absence of an analgesic effect of the implanted prostheses described above. This threshold persisted until the eighth day as it is 42.7 mmHg±1.7 mmHg.

Textile components A, virgin and functionalized with a host polymer coating according to Example 3, loaded with ropivacaine (10 mg/g), were implanted in two groups of 10 rats each. Pain was evaluated one day after implantation up to 8 days. No post-operative deaths were observed.

An improved pain threshold of 46 mmHg was thus observed for the virgin textile component A loaded with ropivacaine (10 mg/g) during the first two days after implantation. A significant pain relief effect was observed for Example 3, loaded with ropivacaine at the same concentration (10 mg/g), during the first four days after implantation, with a threshold of 53±1.71 mmHg demonstrating the contribution of the CD polymer in the release of ropivacaine. At the same amount of ropivacaine, the virgin textile component A and the textile component A of Example 3 do not have the same behavior. The implantable device according to Example 3 is more effective than the virgin textile component A in terms of duration and intensity of pain tolerance.

1. An implantable device comprising a textile component at least partially coated with a host polymer coating, said host polymer coating comprising a polymer of cyclodextrin(s) and/or derivative(s) of cyclodextrin(s) and/or inclusion complex(es) of cyclodextrin and/or inclusion complex(es) of cyclodextrin derivative(s), wherein said textile component comprises multifilament yarns and/or spun yarns made of fibers and wherein at least a part of the filaments and/or fibers each have a diameter of less than or equal to 25 micrometers.

2. The implantable device as claimed in claim 1, wherein the ratio of the mass of the host coating to the total mass of the textile component coated with said host polymer coating is greater than or equal to 10%.

3. The implantable device as claimed in claim 1, wherein the textile component has a surface density of less than or equal to 120 g/m².

4. The implantable device as claimed in claim 1, wherein the textile component comprises multifilament yarns and/or spun yarns made of fibers whose the titer of the filaments and/or the fibers is less than or equal to 10 dtex.

5. The implantable device as claimed in claim 1, wherein the textile component is a knitted component.

6. The implantable device as claimed in claim 1, wherein said device is selected from a list consisting of: a prosthesis for the treatment of a hernia in the abdominal region, a ligament, a prosthesis for the treatment of a prolapse of at least one pelvic organ, a prosthesis for the treatment of male erectile dysfunction, and a prosthesis for the treatment of the spine.

7. The implantable device as claimed in claim 1, wherein the multifilament yarns and/or the spun yarns made of fibers are made of at least one or more polymer(s) chosen from a list consisting of: polyolefins, polymers based on polyethylene oxide (PEO), block copolymers based on polypropylene oxide and ethylene oxide polyesters, and polyamides.

8. The implantable device as claimed in claim 1, wherein the host polymer coating comprises at least one guest functional agent selected from a list comprising: antibiotics, anti-inflammatory agents, anticoagulants, antithrombogenic agents, anti-mitotic agents, anti-proliferation agents, anti-adhesion agents, anti-migration agents, cell adhesion promoters, growth factors, antiparasitic molecules, hormones, antifungals, antimicrobial molecules, antiseptics, and analgesic agents.

9. The implantable device as claimed in claim 1, wherein the textile component comprises openings having at least one dimension greater than or equal to 1 mm.

10. The implantable device as claimed in claim 1, wherein the textile component is selected from a list consisting of: warp or weft knit material, fabrics, braids, twisted elements, or a combination thereof.

11. A process for manufacturing an implantable device, comprising the following steps:
(i) providing a textile component comprising multifilament yarns and/or spun yarns made of fibers, at least a part of the filaments and/or fibers each having a diameter of less than 25 micrometers;
(ii) applying an aqueous solution comprising:
at least one cyclodextrin and/or at least one cyclodextrin derivative and/or at least one inclusion complex of cyclodextrin and/or at least one inclusion complex of cyclodextrin derivative;
at least one (poly)carboxylic acid and/or its (poly)carboxylic acid anhydride,
and optionally at least one catalyst,
on at least a part of the multifilament yarns and/or spun yarns made of fibers;
(iii) applying a heat treatment to the textile component obtained after step (ii) in order to form a host coating comprising a polymer of cyclodextrin(s) and/or derivative(s) of cyclodextrin(s) and/or inclusion complex(es) of cyclodextrin and/or inclusion complex(es) derivative(s) of cyclodextrin(s);
obtaining the implantable device.

12. The manufacturing process as claimed in claim 11, comprising a step of washing (iv) of the textile component after step (iii) during which the textile component is washed with water or an aqueous solution having a temperature greater than or equal to 45° C.

13. The manufacturing process as claimed in claim 11, comprising a step of neutralizing the residual acidity of the host coating.

14. An implantable device as claimed in claim 4, wherein the textile component comprises multifilament yarns and/or spun yarns made of fibers whose the titer of the filaments and/or the fibers is less than or equal to 4 dtex.

15. An implantable device as claimed in claim 15, wherein the textile component is a warp knit component.

16. An implantable device as claimed in claim 16, wherein said device is selected from a list consisting of: a prosthesis for the treatment of a hernia in the inguinal region, a prosthesis for the treatment of a hernia in the femoral region, a prosthesis for the treatment of a hernia in the umbilical region, or a prosthesis for the treatment of a epigastric hernia, and an intervertebral prosthesis.

17. The implantable device as claimed in claim 7, wherein the multifilament yarns and/or the spun yarns made of fibers are made of at least one or more polymer(s) chosen from a list consisting of: (co)polymers of lactic acid, derived from the polymerization of at least L-lactide and/or at least D-lactide and/or at least meso-lactide, polymers derived from the polymerization of at least glycolide, copolymers derived from the polymerization of at least lactide and at least glycolide, block (co)polymers based on polylactide and polyethylene glycol, or a mixture thereof.

18. The implantable device as claimed in claim 8, wherein the host polymer coating comprises at least one guest functional agent selected from a list comprising: ciprofloxacin, ropivacaine, bupivacaine, lidocaine, and levobupivacaine.

19. The implantable device as claimed in claim 8, wherein the host polymer coating comprises at least one guest functional agent that is ropivacaine.

20. A process for manufacturing an implantable device as claimed in claim 1, comprising the following steps:
(i) providing a textile component comprising multifilament yarns and/or spun yarns made of fibers, at least a part of the filaments and/or fibers each having a diameter of less than 25 micrometers;
(ii) applying an aqueous solution comprising:
at least one cyclodextrin and/or at least one cyclodextrin derivative and/or at least one inclusion complex of cyclodextrin and/or at least one inclusion complex of cyclodextrin derivative;
at least one (poly)carboxylic acid and/or its (poly)carboxylic acid anhydride,
and optionally at least one catalyst, on at least a part of the multifilament yarns and/or spun yarns made of fibers;
(iii) applying a heat treatment to the textile component obtained after step (ii) in order to form a host coating comprising a polymer of cyclodextrin(s) and/or derivative(s) of cyclodextrin(s) and/or inclusion complex(es) of cyclodextrin and/or inclusion complex(es) derivative(s) of cyclodextrin(s);
obtaining the implantable device.
